(19) United States
(12) Patent Application Publication
(10) Pub. No.: US 2007/0238956 A1
(43) Pub. Date: Oct. 11, 2007

(54) Imaging device and method for operating an imaging device

(76) Inventors: Gabriel Haras, Mucke (DE); Heinz-Peter Engels, Munchen (DE)

Correspondence Address: HARNESS, DICKEY & PIERCE, P.L.C., P.O.BOX 8910, RESTON, VA, 20195, US

(21) Appl. No.: 11/642,834

(22) Filed: Dec. 21, 2006

(30) Foreign Application Priority Data
Dec. 22, 2005 (DE) 10 2005 061 557.0

(51) Int. Cl. A61B 5/05; A61B 6/00

(52) U.S. Cl. 600/407; 600/431

(57) ABSTRACT

An imaging device is disclosed for preparing sequential slice images of an object. The device includes a radiation source, a detector, a positioning unit and a control unit for controlling the positioning unit and for evaluating the recorded data of the detector. The control unit is set up for carrying out a first contrast agent measurement at a distal position, determining parameters of the contrast agent propagation from the first contrast agent measurement, carrying out a second contrast agent measurement at a proximal position, calculating operating parameters by taking account of the parameters of the contrast agent propagation, and driving the positioning unit in order to record the slice images with the aid of the calculated operating parameters in a fashion triggered by the second contrast agent measurement. A corresponding method for operating the imaging device is also specified.

### PRIORITY STATEMENT

The present application hereby claims priority under 35 U.S.C. §119 on German patent application number DE 10 2005 061 557.0 filed Dec. 22, 2005, the entire contents of which is hereby incorporated herein by reference.

### FIELD

Embodiments of the invention generally relate to an imaging device for preparing sequential slice images of an object. Embodiments of the invention further generally relate to a method for operating such an imaging device.

### BACKGROUND

An imaging device for preparing sequential slice images serves the purpose of obtaining information relating to the interior of the examined object. For example, the slice images can be used to obtain valuable information relating to the position, the size or the structure of internal organs, of bone tissue or of other soft part tissue of a patient. In particular, the sequential slice images can also be converted into a three dimensional display.

Such an imaging device for recording sequential slice images can, for example, be an x ray computer tomograph, a magnetic resonance tomograph, a photon emission computer tomograph or a positron emission tomograph. Such an imaging device can equally well be designed on the basis of ultrasound.

The contrast of the images of the object that are produced by such an imaging device, for example images of a patient, are caused by locally different properties of excitation, absorption, reflection or emission of the examined matter as compared with the radiation, particle radiation or sound waves used by the imaging device. In the case of an x ray machine, the different absorption or attenuation properties of various types of tissue are utilized for providing contrast. Since, for example, bone tissue and soft part tissue differ greatly in said properties, it is possible to analyze the structure of a bone in the body interior of a patient on the basis of the contrast, associated therewith, in the images.

Organs or vessels that do not differ substantially from one another in said properties for the formation of a contrast in the recorded images of surrounding tissue cannot be examined in the conventional way because of the excessively slight contrast that results. For this reason, when examining an organ supplied with blood, for example a heart, a liver or a vessel in the region of the extremities of the patient, a contrast agent is put into the patient's blood circulation before beginning the examination with the aid of the imaging device. The organs examined are imaged with a satisfactorily high contrast against the surrounding tissue because of the contrast agent.

When preparing sequential slice images with the aid of contrast agent administration, it is then necessary to pay heed to the simultaneous presence of the contrast agent. The propagation of the contrast agent is, however, a highly dynamic process and depends strongly on the constitution of the patient. The minute volume plays a role, for example, in this case. In particular, it is also possible for pathological variations in the blood vessels such as, in particular, stenoses or the like to influence the propagation of the contrast agent. Because of the complexity of the contrast agent propagation, and because of the time constants of the imaging device that are to be observed, it is no trivial problem to observe said conditions for recording slice images with high contrast.

It is known to solve this problem by, for example, feeding the contrast agent in as wide a bolus as possible. This provides a sufficiently long time period for recording the sequential slice images. However, a wide contrast agent bolus has disadvantageously constituted an unnecessary burden for the patient.

U.S. Pat. No. 5,459,769 further discloses a method for a computer tomograph with the aid of which it is possible to determine the instant for starting the sequential slice images during a contrast agent examination. In the known method, images are continuously reconstructed to this end at a fixed scanning position of the imaging device, the increasing contrast being calculated therein for previously selected image regions. After detection of the contrast agent, the scanning of the computer tomograph is started on the basis of the information thus obtained.

However, when preparing sequential slice images, in particular over a large region of space, it is not sufficient to fix the starting instant by detecting the arrival of the contrast agent. During the recording of the sequential slice images, the contrast agent can flow out of the zone examined, or else be overtaken by the imaging device.

### SUMMARY

In at least one embodiment of the invention, an imaging device is specified for preparing sequential slice images of an object that enables a high quality of the sequential slice images during a contrast agent examination. In at least one embodiment of the invention, a method is further specified for operating the imaging device that is associated with the same advantages.

An imaging device, in at least one embodiment, is for preparing sequential slice images of an object having a radiation source, having a detector, having a positioning unit and having a control unit for controlling the positioning unit and for evaluating the recorded data of the detector, in which the control unit is set up for the purpose of carrying out a first contrast agent measurement at a distal position, determining parameters of the contrast agent propagation from the first contrast agent measurement, carrying out a second contrast agent measurement at a proximal measurement position, calculating operating parameters by taking account of the parameters of the contrast agent propagation, and driving the positioning unit in order to record the slice images with the aid of the calculated operating parameters in a fashion triggered by the second contrast agent measurement.

The invention, in at least one embodiment, proceeds here in a first step from the consideration that it is necessary to obtain information relating to the contrast agent propagation in order to adapt the sequential slice images to the propagation of the contrast agent. Since the contrast agent propagation depends substantially on the constitution of the respective patient, a first contrast agent measurement is carried out to this end at a distal position, parameters of the contrast agent propagation being determined. If, for example, the aim is to carry out a vessel runoff of a patient, that is to say a sequence of slice images for imaging the vessel structure throughout the patient, it is then possible to select the region of the patient's lower leg as distal position. It is then possible, for example, to record a series of slice images at the distal position in conjunction with a fixed scanning position, and to determine the contrast agent arrival or propagation by evaluating selected image regions.

In a second step, the invention, in at least one embodiment, proceeds from the consideration of using the parameters, obtained from the contrast agent measurement at the distal position, of the contrast agent propagation to set the operating parameters prescribed for the preparation of the sequential slice images in the desired recording region. The operating parameters are adapted by way of this measure to the actual contrast agent propagation when preparing the desired sequential slice images.

In a third step, finally, the invention, in at least one embodiment, proceeds from the consideration of defining the starting instant for beginning to carry out the preparation of the desired sequential slice images with the aid of a second contrast agent measurement at a proximal position. If the contrast agent arrival is detected at a proximal measuring position, for example once again by an evaluation of a series of sequential slice images at a fixed proximal position, the desired preparation of the sequential slice images can be carried out with the aid of the determined operating parameters by driving the positioning unit appropriately.

It is possible, in particular, owing to at least one embodiment of the invention, to take account both of the parameters, obtained from the first contrast agent measurement, of the contrast agent propagation, and of the information obtained from the second contrast agent measurement at a proximal position to calculate the operating parameters in order to drive the positioning unit before beginning the examination, that is to say before beginning the preparation of the sequential slice images in the selected examination zone. Given an appropriate selection of the proximal position, the two sets of information can be processed, and the operating parameters prescribed for the examination can be calculated therefrom, between the instant of the second contrast agent measurement at the proximal position and reaching the scanning position for the examination, and thus starting the preparation of sequential slice images.

The advancing time of the positioning unit or of the object to be examined, for example, can be prescribed as operating parameters. In particular, it is also possible in the case of a rotating radiation source and/or detector to prescribe the so called pitch as an operating parameter, that is to say the ratio of the advance of the positioning unit and the rotation for slice collimation.

It is also possible owing to at least one embodiment of the invention, to ascertain different contrast agent propagations in the extremities by way of the first contrast agent measurement at a distal position. To this end, the imaging device can propose an adaptation of the addition of contrast agent in order to maintain sequential slice images of equal contrast in the two extremities.

Unfalsified information relating to the contrast agent propagation can be obtained from the separate first contrast agent measurement at a distal position. The first contrast agent measurement is, moreover, independent of the propagation rate of the contrast agent or of the possible scanning rate of the imaging device. The positioning unit can already be set at the desired distal position before beginning to examine the contrast agent. The preparation of the desired sequential slice images that is to be carried out is triggered by the second contrast agent measurement at the proximal position, starting from the actual profile of the contrast agent propagation. The desired success with regard to the quality of the sequential slice images to be prepared is thus attained through triggering the measurement with the aid of a real contrast agent measurement while taking account of unfalsified information, obtained from a separate measurement, relating to the contrast agent propagation.

The control unit is advantageously set up for the purpose of carrying out a contrast agent test measurement as a first contrast agent measurement. To this end, the contrast agent for the contrast agent test measurement is used at a lower dose than required for the examination, the result being to reduce the burden on the patient. The information, obtained from this so called contrast agent test bolus, from the first contrast agent measurement at a distal position can be used directly to determine the parameters of the contrast agent propagation.

In a further advantageous refinement of at least one embodiment, the control unit is set up for the purpose of calculating a first arrival time as parameter of the contrast agent propagation. The arrival time includes, for example, information relating to the propagation rate of the contrast agent. This can be taken into account for the preparation of the desired sequential slice images when setting the scanning time, that is to say the rate of advance of the imaging device.

The control unit is advantageously set up, furthermore, for the purpose of calculating a second arrival time from the second contrast agent measurement, and of deducing a movement parameter of the positioning unit from the difference between the two arrival times. The propagation of the contrast agent can easily be deduced from the difference between the two arrival times. When preparing the sequential slice images over a large region of space, a suitable advance rate of the positioning unit, or a suitable pitch value can be deduced from the difference between the arrival times in the distal position and in the proximal position, and so the sequential slice images to be prepared can always be effected given a sufficiently high contrast agent concentration.

In an expedient refinement, the control unit can in this case be set up, furthermore, for the purpose of calculating directly the propagation rate of the contrast agent in the examined region of space, that is to say between distal position and proximal position from the difference between the arrival times, and outputting it. The contrast agent propagation time corresponds in this case to the blood rate of a patient being examined, a knowledge of which is advantageous for other examinations or the like.

In a further advantageous refinement, the control unit is set up for the purpose of determining the time profile of the contrast agent propagation, in particular the instant of the maximum concentration, from the first contrast agent measurement. Particularly in combination with the detection of the first arrival time, it is therefore possible to determine when the contrast agent has a concentration in the respective examination region that is optimum for imaging. Such an optimum concentration can be defined, for example, by reaching a fixed threshold value of the intensity, the attenuation or some other signal used for contrast formation from a selected image region of the series of slice images effected for the contrast agent measurement.

The information obtained from the first contrast agent measurement can be used in order, starting from the second contrast agent measurement, to define the triggering instant at which the actual examination with preparation of the desired sequential slice images is performed. By way of example, to this end the arrival time can be ascertained in each case when a first threshold value of the contrast agent concentration is reached, and the time interval between the arrival time and the maximum value of the contrast agent concentration from the measurement in the distal position is stored. Starting from this stored value, the arrival time for the same threshold value can be determined in the measurement in the proximal position, and then it is possible therefrom to deduce the instant of the maximum in the contrast agent concentration, or to deduce the presence of a concentration required for preparing the respective slice image.

The control unit is advantageously set up for the purpose of deriving a contrast agent protocol from the first contrast agent measurement and outputting it. If, for example, there is ascertained in the extremities a different contrast agent profile that can be ascribed to a stenosis in an extremity, a correspondingly changed contrast agent protocol can then be output. The protocol can be used during the addition of contrast agent and, for example, be of complex design in that the contrast agent is added in multiple phases or in a fashion spread in the time domain. By taking account of the proposed contrast agent protocol, it is then possible in each case to attain equally good contrasts in the sequential slice images in the recordings of the two extremities.

In an advantageous refinement of at least one embodiment, the control unit is set up for the purpose of in each case carrying out a reconstruction of a slice image for the contrast agent measurements. In this case, a slice image is respectively reconstructed in prescribed time intervals at the respectively set distal and proximal positions, and the signal profile from which it is possible to deduce the concentration of the contrast agent in the examination region is tracked.

It is particularly expedient in this case when the control unit is set up to carry out the contrast agent measurements by in each case evaluating the image signals in at least one selected image region of the slice image. The control unit can, in particular, be configured so as to enable a menu-aided processing program in the manual selection of a desired image region in order to track the image signals.

In an example refinement, the imaging device is designed as a computer tomograph.

According to at least one embodiment of the invention, a method is for operating an imaging device having a radiation source, having a detector and having a positioning unit, comprising the following steps:
Carrying out a first contrast agent measurement at a distal position,
determining parameters of the contrast agent propagation from the first contrast agent measurement,
carrying out a second contrast agent measurement at a proximal position,
calculating operating parameters by taking account of the parameters of the contrast agent propagation, and
driving the positioning unit with the aid of the calculated operating parameters in order to record sequential slice images, the driving being triggered by the second contrast agent measurement.

The advantages outlined for the imaging device, in at least one embodiment, are to be carried over, mutatis mutandis, to the method and to the advantageous developments thereof.

### BRIEF DESCRIPTION OF THE DRAWINGS

Example embodiments of the invention are explained in more detail below with the aid of the drawings, in which:

FIG. 1 shows an x ray computer tomograph as an imaging device, in a perspective illustration,

FIG. 2 shows the profile of a method for operating the x ray computer tomograph illustrated in FIG. 1, in sketched fashion, and

FIG. 3 shows a first and a second contrast agent measurement at a distal and, respectively, at a proximal position.

### DETAILED DESCRIPTION OF THE EXAMPLE EMBODIMENTS

The terminology used herein is for the purpose of describing particular embodiments only and is not intended to be limiting of the present invention. As used herein, the singular forms “a”, “an” and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

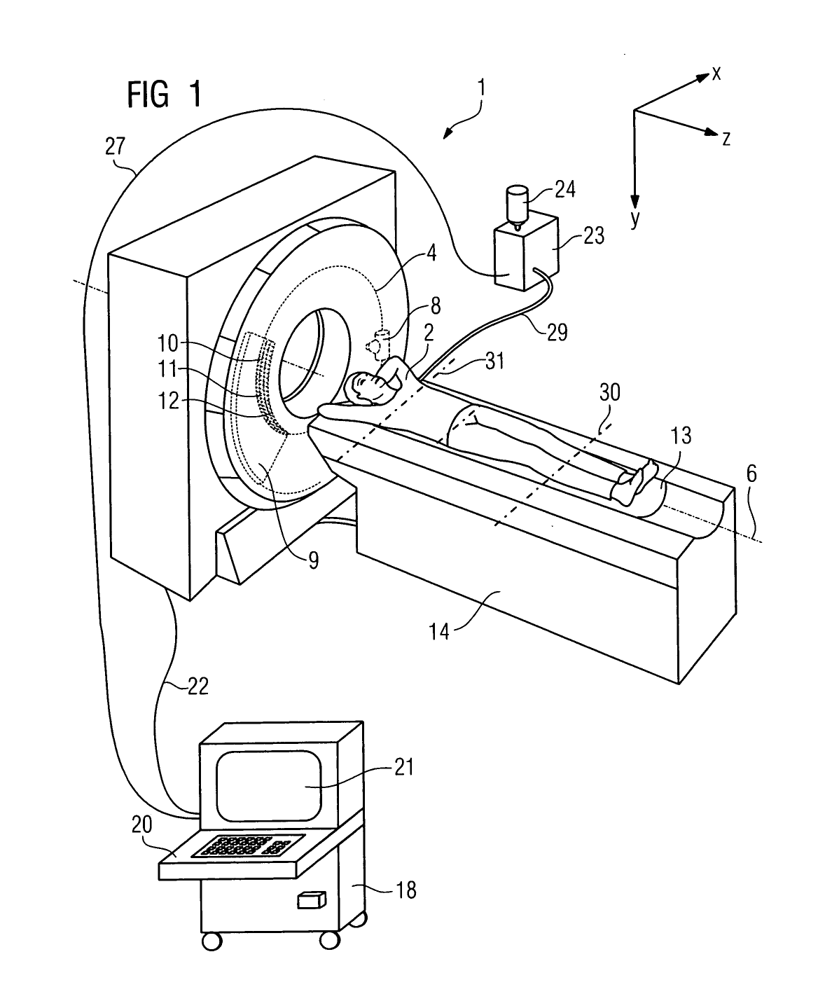

A computer tomograph 1 for examining an object, here a patient 2, is illustrated as imaging device in FIG. 1. The computer tomograph 1 includes a radiation source 8, arranged in a gantry 4 to be able to rotate about an axis of rotation 6, for emitting x radiation. Arranged opposite the radiation source 8 is an arcuate detector 9 that comprises a number of detector elements lined up to form detector rows 10, 11, 12.

The computer tomograph 1 further comprises a table plate 13 that is mounted on a table 14 in a fashion displaceable along the axis of rotation 6.

Recognizable as a further constituent of the computer tomograph 1 is a control unit 18 that has an operator console 20 and a display apparatus 21. The control unit 18 is connected to the computer tomograph 1 via a control line 22.

The control unit 18 is set up for the purpose of driving the advance of the moveable table plate 13 and the rotation of the gantry 4 in order to record sequential slice images. Together, the gantry 4 and table plate 13 form a positioning unit that enables slice images to be recorded at different spatial positions of the patient 2. The scanning rate of the computer tomograph 1 can be set via the pitch value, that is to say the ratio of the advance of the positioning unit and rotation, for the purpose of slice collimation. Here, the rotation of the gantry 4 serves the purpose of recording a slice image at a longitudinal position of the patient 2, whereas the advance of the table plate 13 is responsible for the sequence of the slice images to be recorded.

In order to acquire a slice image, the detector 9 is used to acquire the x radiation emitted by the radiation source 8 and penetrating the patient 2. In the case of the computer tomograph 1 shown, the radiation source 8 generates a fan-shaped x ray beam to this end. A characteristic attenuation image of the x radiation is therefore acquired in each position of the gantry 4. It is possible to reconstruct from the projections obtained in various positions of the gantry 4 a slice image on which tissues having different attenuation properties are displayed with the aid of different gray scale values. The contrast results from the difference between the attenuation properties of adjacent tissues.

By way of example, photodiodes optically coupled to a scintillator or directly converting semiconductors can be used as detector elements of the detector 9.

It is provided to add contrast agent in order to examine organs supplied with blood, for example a heart, a liver or a blood vessel. To this end, the control unit 18 is connected via a control line 27 to a contrast agent device 23 via which a contrast agent 24 can be fed to the patient 2 in a controlled fashion by way of a contrast agent tube 29 in accordance with a prescribed contrast agent protocol.

In order to prepare a sequence of slice images in the thorax region of the patient 2, a contrast agent 24 is firstly administered at a low concentration. Subsequently, a series of slice images are recorded in a fixed distal position 30 and a first contrast agent test measurement is carried out starting from the displayed attenuation values in a selected image region. The contrast agent propagation is deduced from the profile of the attenuation values in the image region. In this process, averaging is carried out for each slice image over the attenuation values in the selected image region, and the resulting profile is used to determine a first arrival time and the instant of reaching a maximum contrast agent concentration. It can also be provided, in particular, to select two image regions in the slice image that are situated in the left leg and in the right one. The contrast agent propagation in the two legs can thereby be tracked separately.

In order to carry out the intended examination of the thorax region of the patient 2, a series of slice images are subsequently recorded in the same way at a proximal position 31. A second contrast agent measurement is carried out as outlined in a selected image region. The contrast agent 24 is fed for this purpose as a care bolus, that is to say at a concentration required for the actual measurement. The proximal region 31 is situated near the proximal beginning of the actually intended examination region. When a prescribed averaged attenuation value derived from the first contrast agent measurement is reached, the actual measurement is started in the selected image region. The information obtained from the first contrast agent measurement in the distal region 30 is converted by the control unit 18 into operating parameters for the positioning unit 4, 13.

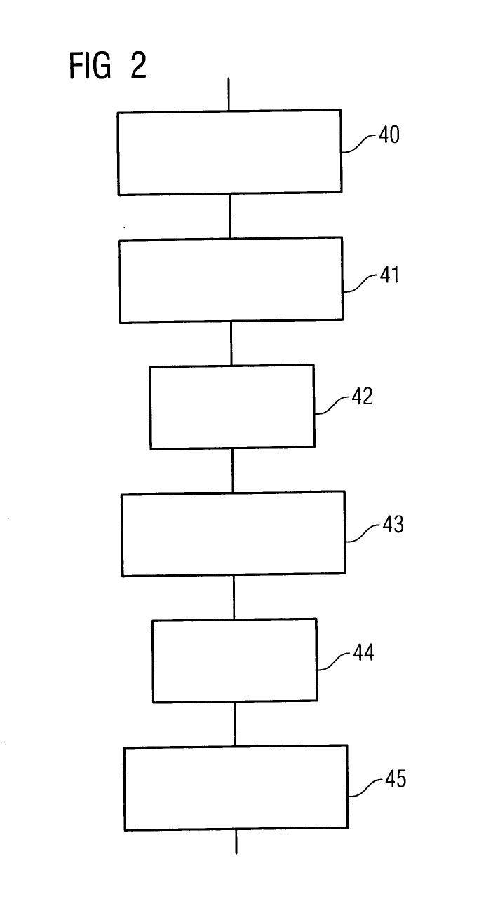

The method for operating the computer tomograph 1 in accordance with FIG. 1 is outlined in FIG. 2. After a single pass topogram of the patient 2, the distal position 30 and the proximal position 31 can be prescribed. The desired scanning region can also be set, for example, for carrying out a vessel runoff of the patient 2. To this end, the control unit 18 can be used to select respectively at the distal position 30 and at the proximal position 31 by way of the display apparatus 21 an image region whose attenuation values are employed to determine the contrast agent propagation.

Alternatively, starting from a selected scanning region the control unit 18 of the computer tomograph 1 can also automatically define the distal position 30 and the proximal position 31.

After definition of the abovementioned parameters, in method step 40 the control unit 18 controls the positioning unit 4, 13 in such a way that slice images can be recorded at the distal position 30. Subsequently, the contrast agent device 23 is driven by way of the control unit 18 to output a test bolus of contrast agent 24. A series of slice images are reconstructed at a fixed distal position 30.

In the subsequent method step 41, the attenuation values in the selected image region are evaluated in order to display a contrast agent profile. That instant at which the attenuation value averaged over the selected image region has been increased by 70 Hounsfield units (HU) is determined as first arrival time, and stored.

In the next method step 42, the positioning unit 4, 13 is driven by the control unit 18 in such a way that slice images can be recorded in the proximal position 31. The control unit 18 controls the contrast agent device 23 so as to add a care bolus. A series of slice images are recorded at the proximal position.

In method step 43, the control unit 18 evaluates the attenuation values in the selected image region of the series of slice images. If an increase in the attenuation value averaged over the selected image region by 70 HU is determined in the selected image region, this instant is determined as second arrival time, and stored. The profile of the first contrast agent measurement is additionally used to evaluate the time remaining starting from the second arrival time up to reaching a concentration of the contrast agent that is required for the examination. Given compliance of the determined delay, the second arrival time triggers the beginning of the actual measurement in the examination region of the vessel runoff.

The difference between the first and second arrival times is determined in method step 44 during the travel time and during the delay time up to the start of the actual measurement. This differential time corresponds to that time which the contrast agent requires from the proximal position 31 to the distal position 30. The optimum scanning time is calculated therefrom for the envisaged preparation of sequential slice images in the thorax region of the patient 2, the sequential slice images always being prepared at an optimum concentration of the contrast agent 24. In other words, the slice images follow the contrast agent propagation.

Finally, the scanning of the computer tomograph 1 over the set scanning region in the thorax region of the patient 2 is started in the last method step 45. To this end, the control unit 18 drives the positioning unit 4, 13 with the aid of the corresponding operating parameters.

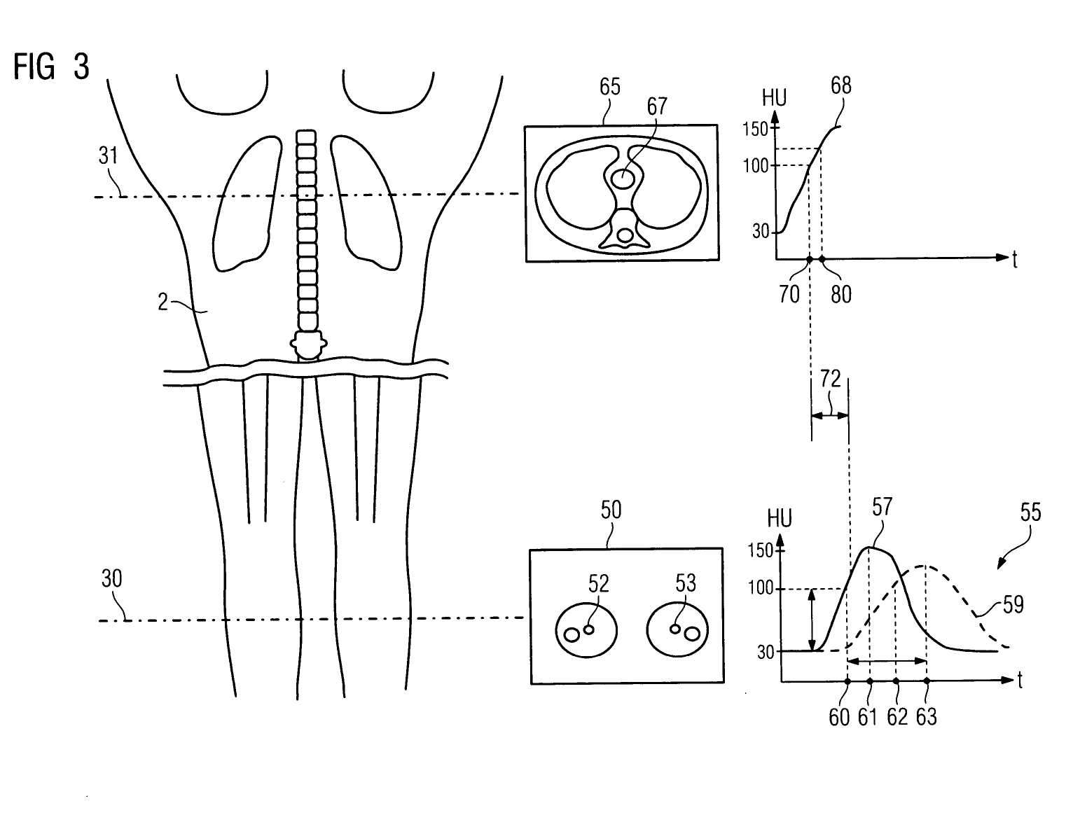

The contrast agent measurements at the distal position 30 and at the proximal position 31 are illustrated by way of explanation in FIG. 3. The distal position 30 lies in the region of the lower leg of the patient 2. The proximal position 31 lies just above the heart of the patient 2.

A contrast agent measurement is carried out at the distal position 30 as first method step with the aid of a test bolus of the contrast agent. To this end, a series of sequential slice images 50 are prepared or reconstructed at the distal position 30. The two extremities of the patient 2 are to be recognized in section in the illustrated slice image 50.

The image regions 52 and 53, which respectively contain a blood vessel of the right leg and of the left one are selected for the first contrast agent measurement. For the purpose of the contrast agent measurement, averaging is carried out in each case over the gray scale values inside 1 are displayed the selected image regions 52 and 53, respectively, for each of the recorded slice images. After introduction of a test bolus of the contrast agent 24, this evaluation produces the illustrated contrast agent measurement 55.

In the illustration of the contrast agent measurement 55 time is displayed along the X-axis, and the attenuation values 1 in Hounsfield units (HU) along the Y-axis. It is to be seen that the first attenuation profile 57 resulting from the evaluation of the first image region 52 rises starting from a constant base value of 30 HU to an attenuation value of 150 HU and subsequently drops. The second attenuation profile 59, which results from evaluating the second image region 53, is delayed in time in relation to the first attenuation profile 57. This can be ascribed, for example, to a stenosis in the left leg of the patient 2. Because of the constricted blood vessels, the contrast agent 24 to this extent reaches the distal position 30 later in the left leg than in the right leg.

The control unit 18 evaluates both the first and the second attenuation profile 57 and 59, respectively. A first arrival time 60 and a peak time 61 of the right leg are determined from the first attenuation profile 57. The first arrival time 60 is taken in this case from the first attenuation profile 57 when a rise in attenuation by 70 HU is reached. A first arrival time 62 and a peak time 63 of the left leg are taken in a similar way from the second attenuation profile 59.

The control unit 18 uses the difference between the two first arrival times 60 and 62 of the right leg and of the left one, respectively, to determine a contrast agent protocol that is output and/or taken into account when carrying out the desired examination. It is taken into account in this contrast agent protocol that the arrival times 60, 62 of the contrast agent 24 differ from one another in the two legs. The prepared contrast agent protocol correspondingly includes a contrast agent bolus that is spread in the time domain.

After the described parameters of the contrast agent propagation have been determined from the first contrast agent measurement, the control unit controls the positioning unit 4, 13 into that position in which the computer tomograph 1 can carry out the second contrast agent measurement at the proximal measuring position 31. A series of slice images 65 are undertaken, in turn, at the defined distal position 31. As already described, a selected image region 67 is prescribed whose gray scale values are used to determine an attenuation profile 68 at the proximal position 31. A care bolus was correspondingly introduced by driving the contrast agent device 23 appropriately before beginning to prepare the series of slice images 65.

The attenuation profile 68 is monitored in real time by evaluating the selected image region 67 continuously. If the observed attenuation 68 increases by 70 HU, the second arrival time 70 is determined therefrom. A difference 72 is formed between the first arrival time 60 and second arrival time 70. This difference 72 is a measure of the scanning time to be set for the computer tomograph 1. The advance of the table plate 13 and the rotation of the gantry 4 are prescribed by way of the differential time 72 in accordance with the envisaged scanning region.

After a time difference, which can also be prescribed by the system, an actual measurement is begun at the beginning of scanning 80. The calculation of the operating parameters to be set can be performed by the control unit 18 in the time between the second arrival time 70 and the beginning of scanning 80. Since the contrast agent profile is, for example, known from the first attenuation profile 57 from the test bolus measurement, it is possible to wait over this time between the second arrival time 70 and the beginning of scanning 80. Specifically, once the second arrival time 70 has been ascertained the control unit 18 is aware of when, for example, the attenuation value of 120 HU that is advantageous for preparing the sequential slice images is reached.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An imaging device for preparing sequential slice images of an object, comprising:
a radiation source;
a detector; a positioning unit; and
a control unit for controlling the positioning unit and for evaluating the recorded data of the detector, the control unit being set up for:
carrying out a first contrast agent measurement at a distal position,
determining parameters of the contrast agent propagation from the first contrast agent measurement,
carrying out a second contrast agent measurement at a proximal position,
calculating operating parameters by taking account of the parameters of the contrast agent propagation, and
driving the positioning unit in order to record the slice images with the aid of the calculated operating parameters in a fashion triggered by the second contrast agent measurement.

2. The imaging device as claimed in claim 1, wherein the control unit is further set up for carrying out a contrast agent test measurement as a first contrast agent measurement.

3. The imaging device as claimed in claim 1, wherein the control unit is further set up for calculating a first arrival time as parameter of the contrast agent propagation.

4. The imaging device as claimed in claim 3, wherein the control unit is further set up for calculating a second arrival time from the second contrast agent measurement, and for deducing a movement parameter of the positioning unit from the difference between the two arrival times.

5. The imaging device as claimed in claim 4, wherein the control unit is further set up for calculating the propagation rate of the contrast agent from the difference between the arrival times, and outputting it.

6. The imaging device as claimed in claim 1, wherein the control unit is further set up for determining the time profile of the contrast agent propagation from the first contrast agent measurement.

7. The imaging device as claimed in claim 1, wherein the control unit is further set up for deriving a contrast agent protocol from the first contrast agent measurement and outputting it.

8. The imaging device as claimed in claim 1, wherein the control unit is further set up for, in each case, carrying out a reconstruction of a slice image for the contrast agent measurements.

9. The imaging device as claimed in claim 8, wherein the control unit is further set up for carrying out the contrast agent measurements by, in each case, evaluating at least one selected image region of the slice image.

10. The imaging device as claimed in claim 1, wherein the imaging device is designed as a computer tomograph.

11. A method for operating an imaging device including a radiation source, a detector and a positioning unit, comprising:
carrying out a first contrast agent measurement at a distal position;
determining parameters of the contrast agent propagation from the first contrast agent measurement;
carrying out a second contrast agent measurement at a proximal position;
calculating operating parameters by taking account of the parameters of the contrast agent propagation; and
driving the positioning unit with the aid of the calculated operating parameters in order to record sequential slice images, the driving being triggered by the second contrast agent measurement.

12. The method as claimed in claim 11, wherein a contrast agent test measurement is carried out as a first contrast agent measurement.

13. The method as claimed in claim 11, wherein a first arrival time is calculated as parameter of the contrast agent propagation.

14. The method as claimed in claim 13, wherein a second arrival time is calculated from the second contrast agent measurement, and a movement parameter of the positioning unit is deduced from the difference between the two arrival times.

15. The method as claimed in claim 14, wherein the propagation rate of the contrast agent is calculated from the difference between the arrival times and output.

16. The method as claimed in claim 11, wherein the time profile of the contrast agent propagation is determined from the first contrast agent measurement.

17. The method as claimed in claim 11, wherein a contrast agent protocol is derived from the first contrast agent measurement and output.

18. The method as claimed in claim 11, wherein, in each case, a reconstruction of a slice image is carried out for the contrast agent measurements.

19. The method as claimed in claim 18, wherein the contrast agent measurements are carried out in each case by evaluating at least one selected image region of the slice image.

20. The method as claimed in claim 11, wherein the method is for operating a computer tomograph.

21. The imaging device as claimed in claim 2, wherein the control unit is set up for the purpose of calculating a first arrival time as parameter of the contrast agent propagation.

22. The imaging device as claimed in claim 21, wherein the control unit is further set up for calculating a second arrival time from the second contrast agent measurement, and for deducing a movement parameter of the positioning unit from the difference between the two arrival times.

23. The imaging device as claimed in claim 1, wherein the control unit is further set up for determining the time profile of the maximum concentration from the first contrast agent measurement.

24. The method as claimed in claim 12, wherein a first arrival time is calculated as parameter of the contrast agent propagation.

25. The method as claimed in claim 11, wherein the time profile of the instant of the maximum concentration is determined from the first contrast agent measurement.

26. A computer readable medium including program segments for, when executed on a computer device, causing the computer device to implement the method of claim 11.
